(19) United States
(12) Patent Application Publication
(10) Pub. No.: US 2010/0151037 A1
(43) Pub. Date: Jun. 17, 2010

(54) METHOD FOR THE PREPARATION OF NANOPARTICLES CONTAINING A POORLY WATER-SOLUBLE PHARMACEUTICALLY ACTIVE COMPOUND

(76) Inventors: Yivan Jiang, Stow, MA (US); Zhiwei Jiang, Stow, MA (US)

Correspondence Address: WILMERHALE/BOSTON, 60 STATE STREET, BOSTON, MA, 02109, US

(21) Appl. No.: 12/536,576

(22) Filed: Aug. 6, 2009

Related U.S. Application Data
(60) Provisional application No. 61/086,859, filed on Aug. 7, 2008.

(51) Int. Cl. A61K 9/14; A61K 31/235; A61P 35/04

(52) U.S. Cl. 424/499; 424/489; 424/501; 424/500; 514/545

(57) ABSTRACT

The present application relates to a method for preparing nanoparticles containing a poorly water-soluble pharmaceutically acceptable compound and compositions containing such nanoparticulates.

### CROSS REFERENCE TO RELATED APPLICATIONS

This application claims the benefit of priority under 35 U.S.C. §119(e) to U.S. patent application Ser. No. 61/086,859 filed on Aug. 7, 2008 and is hereby incorporated by reference in its entirety.

### TECHNICAL FIELD

The present application relates to a method for preparing nanoparticles containing a poorly water-soluble pharmaceutically active compound and compositions containing such nanoparticles.

### BACKGROUND

There is a critical need in the pharmaceutical industries to formulate a poorly water-soluble pharmaceutically active compound into formulations suitable for oral, injectable, or other routes of delivery. Nanoparticle formulations containing the poorly water-soluble pharmaceutically active compound provide advantages such as improved oral bioavailability, reduced in vivo variability, favorable toxicity profile of injectable formulations (e.g., due to the reduced use of organic solvents), passive targeting of certain cancerous tumors associated with loose fenestrated vasculature across which small drug particles can directly migrate, as well as sustained release form of intramuscular injectable drugs which form is otherwise not available to drugs having low bioavailability.

Methods for preparing microparticles and nanoparticles are disclosed in, for example, U.S. Pat. No. 5,145,684; U.S. Pat. No. 6,604,698; Xu et al., Pharm. Research, 1990, vol. 7(5), 553-557, U.S. Pat. No. 6,835,396; U.S. Pat. No. 2,745,785; U.S. Pat. No. 4,826,689; U.S. Pat. No. 4,997,454; U.S. Pat. No. 5,118,528; U.S. Pat. No. 5,780,062; U.S. Pat. No. 6,143,211; U.S. Pat. No. 6,235,224; U.S. Pat. No. 6,607,784; U.S. Pat. No. 6,869,617; U.S. Pat. No. 6,884,436; U.S. Pat. No. 6,951,656; U.S. Pat. No. 6,977,085; U.S. Pat. No. 7,037,528; U.S. Pat. No. 7,193,084; U.S. Pat. No. 6,623,761; U.S. Pat. No. 6,682,758; U.S. Pat. No. 6,756,062; U.S. Pat. No. 6,974,593; WO 9713503; U.S. Pat. No. 7,314,516; U.S. Pat. No. 5,766,635; U.S. Pat. No. 5,716,642; U.S. Pat. No. 5,665,331; U.S. Pat. No. 5,662,883; U.S. Pat. No. 5,560,932; U.S. Pat. No. 4,608,278; U.S. Pat. No. 5,133,908; U.S. Pat. No. 5,188,837; U.S. Pat. No. 5,700,471; and U.S. Pat. No. 6,682,761.

Casein is the predominant phosphoprotein (αS1, αS2, β, κ) that accounts for nearly 80% of proteins in milk and cheese. Casein is relatively hydrophobic, making it poorly soluble in water. It is found in milk as a suspension of particles called casein micelles which show some resemblance with surfactant-type micellae in a sense that the hydrophilic parts reside at the surface.

WO 2007122613 discloses a re-assembled casein micelle comprising at least one exogenous hydrophobic biologically active compound within the micelle. WO 2008065502 relates to compositions comprising nanoparticles comprising a low-solubility drug and an enteric polymer as matrix, and casein or a pharmaceutically acceptable form thereof. U.S. Pat. No. 3,995,070 discloses a process for preparing a casein micelle.

Coenzyme Q10 is a benzoquinone, where Q refers to the quinone chemical group, and 10 refers to the isoprenyl chemical subunits. It is a component of the electron transport chain and participates in aerobic cellular respiration, generating energy in the form of ATP. Ninety-five percent of the human body's energy is generated this way. Coenzyme Q10 as a nutrient supplement has been recommended for congestive heart failure, cardiac arrhythmias, ischemic injury, Parkinson's disease, mitochondrial cytopathies, and chronic fatigue. Coenzyme Q10, however, is poorly soluble in water. In dry powder form, its bioavailability is very poor, ranging as low as three percent.

Fibrates are a group of drugs which are known as hypolipidaemic agents. They include bezafibrate, cipprofibrate, fenofibrate and gemfibrizol. Fenofibrate is the most used fibrate and has been extensively studied in formulation. Fibrates have the beneficial effect of lowering triglyceride and cholesterol levels in the blood and hence reducing the risk of coronary heart disease. However, fibrates are poorly water-soluble and have low bioavailability. Furthermore, fibrates can have big bioavailability difference between in fasted and in fed conditions, which can result in complications in clinical setting.

There remains a need to develop a method for preparing nanoparticles containing a poorly water-soluble pharmaceutically acceptable compound and compositions containing such nanoparticles.

### SUMMARY

In one aspect, the present invention provides a method for the preparation of nanoparticles containing a poorly water-soluble pharmaceutically active compound. The method comprises:
mixing the compound and at least one surfactant in a water-miscible organic solvent to form a solution;
infusing water and optionally an additional surfactant to the solution while homogenizing the solution to form a suspension;
optionally adding at least one co-surfactant and/or bulking agent to the suspension while homogenizing the suspension; and
drying the suspension to provide nanoparticles containing the poorly water-soluble pharmaceutically active compound having a particle size in the range from about 50 nm to about 5000 nm, wherein said drying is achieved by spray drying, roto-vap evaporation, or freeze drying.

In another aspect, the present invention provides a nanoparticle containing a poorly water-soluble pharmaceutically active compound prepared according to the method as described herein.

In yet another aspect, the present invention provides a pharmaceutical composition comprising the nanoparticle prepared according to the method as described herein and a pharmaceutically acceptable carrier.

In a further aspect, the present invention provides a composition comprising:
about 1-60% by weight nanoparticles of a pharmaceutically active compound;
about 5-90% by weight at least one surfactant and at least one co-surfactant which are on the surface of the nanoparticles;
about 0-90% by weight a bulking agent; and
about 0-5% by weight water.

In another aspect, the present invention provides a composition comprising:
about 1-60% by weight nanoparticles of coenzyme Q10;
about 5-90% by weight at least one surfactant and at least one co-surfactant which are on the surface of the nanoparticles;
about 0-90% by weight a bulking agent; and
about 0-5% by weight water.

In yet another aspect, the present invention provides a composition comprising;
about 5-60% by weight nanoparticles of fibrate;
about 5-90% by weight at least one surfactant and at least one co-surfactant which are on the surface of the nanoparticles; and
about 0-90% by weight a bulking agent;
about 0-5% by weight water.

### BRIEF DESCRIPTION OF THE DRAWINGS

FIG. 1 is a schematic diagram illustrating one embodiment of the process as described in Examples 1 and 2.

FIG. 2 shows a SEM morphology of nanoparticulate coenzyme Q10 composition prepared as described in example 1.

FIG. 3 shows a SEM morphology of nanoparticulate fenofibrate composition prepared as described in Example 2.

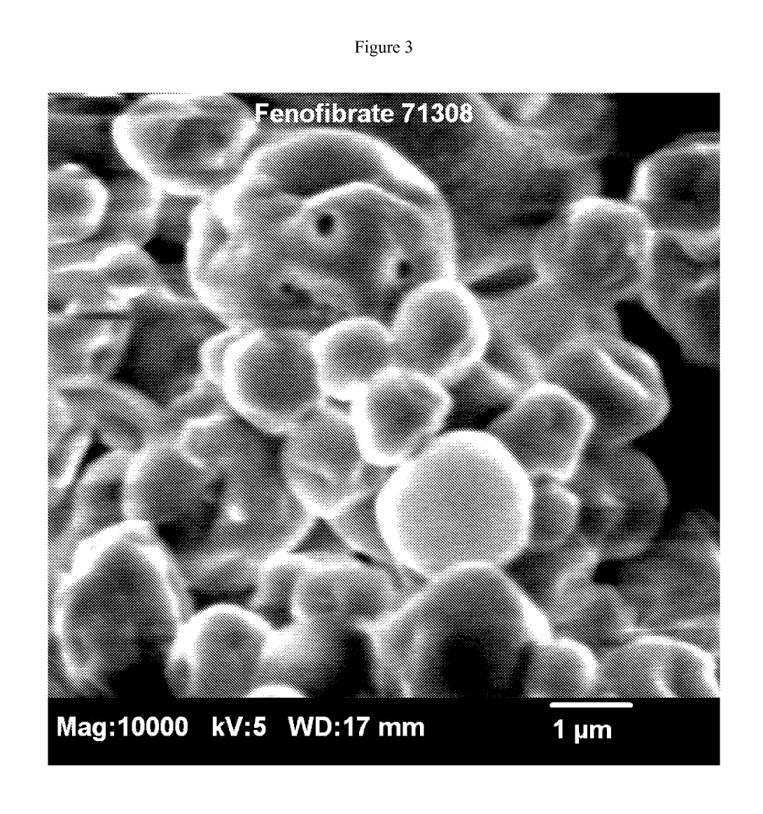

### DETAILED DESCRIPTION

The term “precipitation” used herein means formation of a new solid phase in a continuous liquid phase or formation of a new liquid phase in a continuous liquid phase.

The term “water” used herein means pure water, e.g., ionized water. The term “water” also includes aqueous solution, including, but not limited to, saline solution, dextrose solution, and other aqueous solutions containing at least one pharmaceutically acceptable salt and/or at least one pharmaceutically acceptable surfactant.

Non-limiting examples of “casein derivatives” used herein include milk, fat reduced milk, skim milk, milk powder, pharmaceutically acceptable salts of casein, enzymatically hydrolyzed casein, as well as chemically modified caseins such as chemically superphosphorylated casein and lysine residue partially alkylated casein.

The term “spray drying” used herein refers to a method of drying a liquid feed through a hot gas. The liquid feed is pumped through an atomizer device that produces fine droplets into a main drying chamber.

The term “rotovap evaporation” used herein refers to a method of drying or condensing a liquid in a round bottom flask through evaporation using rotary evaporator which is designed to allow you to distill a liquid under conditions of reduced pressure.

The term “median particle size” refers to the particle diameter at which the cumulative volume of the finer particles reaches 50% of the total volume of all particles.

The present invention provides, in part, a method for the preparation of nanoparticles containing a poorly water-soluble pharmaceutically active compound. The method comprises: mixing the compound and at least one surfactant in a water-miscible organic solvent to form a solution; infusing water and optionally an additional surfactant to the solution while homogenizing the solution to form a suspension; optionally adding at least one co-surfactant and/or bulking agent to the suspension while homogenizing the suspension; and drying the suspension to provide nanoparticles containing the poorly water-soluble pharmaceutically active compound having a particle size in the range from about 50 nm to about 5000 nm. The drying step can be achieved by spray drying, rotovap evaporation, or freeze drying.

In certain embodiments, the water-miscible organic solvent includes acetic acid, acetone, methanol, ethanol, 1-propanol , 2-propanol, formic acid, propionic acid, dimethylformamide, 1,4-dioxane, tetrahydrofuran, N-methyl-2-pyrrolidinone, 2-pyrrolidone, dimethyl sulfoxide, dimethylacetamide, ethylene glycol, propylene glycol and mixtures thereof. In certain other embodiments, the water-miscible organic solvent includes the acetic acid, acetone, methanol, ethanol, 1-propanol , 2-propanol, formic acid and mixtures thereof. The selected solvents provide advantages such as: (a) good solubility for the pharmaceutically active compound or compound mixture; (b) low toxicity; and (c) low boiling point.

In certain embodiments, the at least one surfactant includes glycerol mono-(or di-) fatty acid esters, lecithin, phospholipids (such as phosphatidyl choline, phosphatidyl ethanolamine, phosphatidyl inositol, sphingomyelin, and the like), cholesterol, PEG-phospholipids, PEG-cholesterol, PEG-cholesterol derivatives, PEG-vitamin A, PEG-vitamin E, PEG-glycerol mono-(or di-)fatty acid esters, ethylene glycol mono-fatty acid esters, propylene glycol mono-fatty acid esters, 3-dialkyl(C1-8)amino-propylene glycol di-fatty acid esters, poly(ethylene glycol) mono-fatty acid esters, stearic acid, sorbitan esters, polyoxyethylene alkyl ethers, polyoxyethylene castor oil derivatives, polyoxyethylene sorbitan fatty acid esters, polyvinyl alcohol, polyvinylpyrrolidone, poloxamers; poloxamines, mixtures of sucrose stearate and sucrose distearate, random copolymers of vinyl acetate and vinyl pyrrolidone, deoxycholic acid, glycodeoxycholic acid, taurocholic acid and mixtures thereof.

In certain embodiments, the infused water may include aqueous solution such as saline solution, dextrose solution, buffers, and other aqueous solutions containing at least one pharmaceutically acceptable salt and/or at least one pharmaceutically acceptable surfactant, besides pure water, e.g., ionized water.

In certain embodiments, the volume of the water infused is in the range from about 3 to about 200 times of the volume of the water-miscible organic solvent.

Generally, slower flow rate of water is preferred for generating smaller particles. In some cases, the mild mechanic agitator like food preparation blender (such as Dynamic Mixer MD95, 2301 Sturgis Rd., Oxnard, Calif. 93030) can produce good results. Other typical mechanic agitators can also be sued, for example, high shear mixer such as the mixers produced by Silverson Machines, Inc. (East Longmeadow, Mass., USA) and high pressure homogenizer such as the machines produced by Avestin Inc. (Ottawa, Canada), as well as sonicator. The step of mixing the compound and at least one surfactant in a water-miscible organic solvent to form a solution can be performed at a temperature range from 0° C. to 110° C., preferably at a range above melting point of the pharmaceutically active compound but below the boiling point of the water miscible organic solvent. It is also preferred to minimize the temperature difference between the water and the compound solution (or suspension) before infusion.

In certain embodiments, the additional surfactant and the co-surfactant each independently includes: anionic biopolymers (such as casein or its derivative), anionic polymers, cationic biopolymers, salts of these acids (deoxycholic acid, glycocholic acid, glycodeoxycholic acid, taurocholic acid), glycerol mono-(or di-)fatty acid esters, lecithin, phospholipids (such as phosphatidyl choline, phosphatidyl ethanolamine, phosphatidyl inositol, sphingomyelin, and the like), cholesterol, PEG-phospholipids, PEG-cholesterol, PEG-cholesterol derivatives, PEG-vitamin A, PEG-vitamin E, PEG-glycerol mono-(or di-)fatty acid esters, ethylene glycol mono-fatty acid esters, propylene glycol mono-fatty acid esters, 3-dialkyl(C1-8)amino-propylene glycol di-fatty acid esters, poly(ethylene glycol) mono-fatty acid esters, stearic acid, sorbitan esters, polyoxyethylene alkyl ethers, polyoxyethylene castor oil derivatives, polyoxyethylene sorbitan fatty acid esters, polyvinyl alcohol, polyvinylpyrrolidone, poloxamers; poloxamines, mixtures of sucrose stearate and sucrose distearate, random copolymers of vinyl acetate and vinyl pyrrolidone, deoxycholic acid, glycodeoxycholic acid, taurocholic acid and mixtures thereof. In certain other embodiments, the additional surfactant and the co-surfactant each independently includes anionic biopolymers (such as casein or its derivative), anionic polymers, cationic biopolymers and mixtures thereof.

The optional addition of co-surfactant(s) and bulking reagent(s) is to further stabilize the nanoparticles and prevent the nanoparticles from aggregation during the next evaporation step.

In certain embodiments, the bulking agent includes starches or its derivatives, mannitol, lactose, maltitol, maltodextrin, maltose, dextrates, dextrin, dextrose, fructose, sorbitol, glucose, sucrose, carboxymethylcellulose, hydroxypropylcellulose, microcrystalline cellulose, ethylcellulose, methylcellulose, other suitable cellulose derivatives, gelatin, alginic acid, and its salt, colloidal silicon dioxide, croscarmellose sodium, crospovidone, magnesium aluminum silicate, povidone, benzyl phenylformate, chlorobutanol, diethyl phthalate, calcium stearate, glyceryl palmitostearate, magnesium oxide, poloxamer, polyvinyl alcohol, sodium benzoate, sodium lauryl sulfate, sodium stearyl fumarate, stearic acid, talc, zinc stearate, acacia, acrylic and methacrylic acid co-polymers, gums such as guar gum, milk derivatives such as whey, pharmaceutical glaze, glyceryl palmitostearate, hydrogenated vegetable oil, kaolin, magnesium carbonate, magnesium oxide, polymethacrylates, sodium chloride and mixtures thereof.

The drying step of the nanoparticle suspension can be achieved by spray drying, roto-vap evaporation, or freeze drying. There are a number of advantages of drying the nanoparticle suspension, including but are not limited to: (1) stabilizing the nanoparticles against particle aggregation or flocculation by reducing particle mobility in a solid state; (2) stabilizing the nanoparticles against Ostwald ripening resulting from changes in solubility due to temperature fluctuation (Luckham, Pestic. Sci., 1999, 25, 25-34) by depleting solvent; (3) facilitating next formulation step for solid dosage forms; and (4) removing toxic organic solvent involved in nanoparticle preparation.

In certain embodiments, the nanoparticles prepared according to the methods described herein have a median particle size less than about 3000 nm. In certain other embodiments, the nanoparticles have a median particle size less than about 2500 nm. In yet other embodiments, the nanoparticles have a median particle size less than about 1000 nm.

Applicants surprisingly found that the combination of casein or its derivative and glycerol mono-(or di-)fatty acid ester and/or phospholipids provides significantly reduced nanoparticle aggregation or agglomeration during the evaporation process of the aqueous nanoparticle suspension. Accordingly, this combination can be used in the preparation of nanoparticles of various pharmaceutically active compounds. Substitutions of casein or its derivative with same weight amount of polyvinylpyrrolidone, or phospholipids, or starche, or mannitol, or lactose, or sorbitol, or glucose result in significant aggregation of nanoparticles and less re-dispersibility of the nanoparticulate compositions.

In certain embodiments, the poorly water-soluble pharmaceutically active compound is coenzyme Q10. In certain other embodiments, the at least one surfactant is a phospholipid. In certain other embodiments, the phospholipid is lecithin. In certain other embodiments, the additional surfactant and the co-surfactant are each independently sodium caseinate.

In certain embodiments, the poorly water-soluble pharmaceutically active compound is fenofibrate. In certain other embodiments, the at least one surfactant is glycerol mono-oleate. In certain other embodiments, the additional surfactant and the co-surfactant are each independently sodium caseinate.

The present invention provides, in part, a nanoparticle containing a poorly water-soluble pharmaceutically active compound prepared according to the methods as described herein.

The present invention provides, in part, a pharmaceutically composition comprising the nanoparticles prepared according to the methods as described herein, and a pharmaceutically acceptable carrier.

The present invention provides, in part, a composition comprising:
about 1-60% by weight nanoparticles of a pharmaceutically active compound;
about 5-90% by weight at least one surfactant and at least one co-surfactant which are on the surface of the nanoparticles;
about 0-90% by weight a bulking agent; and
about 0-5% by weight water.

In certain embodiments, the at least one co-surfactant is casein or its derivatives. In certain other embodiments, the at least one surfactant is selected from anionic biopolymers (excluding casein or its derivative), anionic polymers, cationic biopolymers, salts of these acids (deoxycholic acid, glycocholic acid, glycodeoxycholic acid, taurocholic acid), glycerol mono-(or di-)fatty acid esters, lecithin, phospholipids (such as phosphatidyl choline, phosphatidyl ethanolamine, phosphatidyl inositol, sphingomyelin, and the like), cholesterol, PEG-phospholipids, PEG-cholesterol, PEG-cholesterol derivatives, PEG-vitamin A, PEG-vitamin E, PEG-glycerol mono-(or di-)fatty acid esters, ethylene glycol mono-fatty acid esters, propylene glycol mono-fatty acid esters, 3-dialkyl(C1-8)amino-propylene glycol di-fatty acid esters, poly(ethylene glycol) mono-fatty acid esters, stearic acid, sorbitan esters, polyoxyethylene alkyl ethers, polyoxyethylene castor oil derivatives, polyoxyethylene sorbitan fatty acid esters, polyvinyl alcohol, polyvinylpyrrolidone, poloxamers; poloxamines, mixtures of sucrose stearate and sucrose distearate, random copolymers of vinyl acetate and vinyl pyrrolidone, deoxycholic acid, glycodeoxycholic acid, and taurocholic acid and mixtures thereof. In certain embodiments, the at least one surfactant is selected from glycerol mono-(or di-)fatty acid esters, lecithin, phospholipids, and mixtures thereof. In certain other embodiments, the at least one surfactant is selected from glycerol mono-(or di-)fatty acid esters excluding glycerol mono-stearate.

The present invention provides, in part, a composition comprising:
about 1-60% by weight nanoparticles of coenzyme Q10;
about 5-90% by weight at least one surfactant and at least one co-surfactant which are on the surface of the nanoparticles;
about 0-90% by weight a bulking agent; and
about 0-5% by weight water.

In certain embodiments, the coenzyme Q10 is in a form selected from the group consisting of a crystalline phase, an amorphous, a semi-crystalline phase, a semi-amorphous, and mixtures thereof. In certain other embodiments, the size of the nanoparticles is in the range selected from the group consisting of less than about 5 μm, less than about 3 μm, less than about 1.5 μm, less than about 1 μm, less than about 900 nm, less than about 800 nm, less than about 700 nm, less than about 600 nm, less than about 500 nm, less than about 400 nm, less than about 300 nm, less than about 200 nm, and less than about 100 nm.

In certain embodiments, the at least one surfactant and at least one co-surfactant are independently selected from glycerol mono-(or di-)fatty acid esters, lecithin, phospholipids (such as phosphatidyl choline, phosphatidyl ethanolamine, phosphatidyl inositol, sphingomyelin, and the like), cholesterol, PEG-phospholipids, PEG-cholesterol, PEG-cholesterol derivatives, PEG-vitamin A, PEG-vitamin E, PEG-glycerol mono-(or di-)fatty acid esters, ethylene glycol mono-fatty acid esters, propylene glycol mono-fatty acid esters, 3-dialkyl(C1-8)amino-propylene glycol di-fatty acid esters, poly(ethylene glycol) mono-fatty acid esters, stearic acid, sorbitan esters, polyoxyethylene alkyl ethers, polyoxyethylene castor oil derivatives, polyoxyethylene sorbitan fatty acid esters, polyvinyl alcohol, polyvinylpyrrolidone, poloxamers, poloxamines, mixtures of sucrose stearate and sucrose distearate, random copolymers of vinyl acetate and vinyl pyrrolidone, deoxycholic acid, glycodeoxycholic acid, taurocholic acid, and the group consisting of anionic biopolymers (such as casein or its derivative), anionic polymers, cationic biopolymers and mixtures thereof. In certain embodiments, the at least one surfactant and the at least one co-surfactant are independently selected from the group consisting of glycerol mono- (or di-)fatty acid esters, lecithin, phospholipids (such as phosphatidyl choline, phosphatidyl ethanolamine, phosphatidyl inositol, sphingomyelin, and the like), PEG-phospholipids, PEG-vitamin E, PEG-glycerol mono-(or di-)fatty acid esters, ethylene glycol mono-fatty acid esters, propylene glycol mono-fatty acid esters, di-fatty acid esters, poly(ethylene glycol) mono-fatty acid esters, the group consisting of anionic biopolymers (such as casein or its derivative) and mixtures thereof.

In certain embodiments, the at least one surfactant includes glycerol mono-(or di-)fatty acid esters, lecithin, phospholipids and mixtures thereof; and the at least one co-surfactant is casein or its derivatives. In certain other embodiments, the at least one surfactant is lecithin and the at least one co-surfactant is sodium caseinate.

The present invention provides, in part, a solid powder containing Coenzyme Q10 nanoparticles which has good stability, high concentration and which can be prepared at reasonable cost useful as nutrient supplements. High concentration of Coenzyme Q10 in the prepared solid powder (20-45% by weight) may also facilitate the process transforming the powder into an orally administerable dosage form such as capsule, tablet, powder, and liquid beverage. The powder prepared according the methods described herein can also be further processed into a cream for cosmetic use.

The present invention provides, in part, a composition comprising;
about 5-60% by weight nanoparticles of fibrate;
about 5-90% by weight at least one surfactant and at least one co-surfactant which are on the surface of the nanoparticles; and
about 0-90% by weight a bulking agent; and
about 0-5% by weight water.

In certain embodiments, the fibrate is fenofibrate. In certain other embodiments, over 50% of the fenofibrate is in a form of amorphous phase. In certain other embodiments, the size of the nanoparticles is in the range selected from the group consisting of less than about 5 μm, less than about 3 μm, less than about 1.5 μm, less than about 1 μm, less than about 900 nm, less than about 800 nm, less than about 700 nm, less than about 600 nm, less than about 500 nm, less than about 400 nm, less than about 300 nm, less than about 200 nm, and less than about 100 nm.

In certain embodiments, the at least one co-surfactant is casein or its derivatives. In certain other embodiments, the at least one surfactant is selected from glycerol mono-(or di-)fatty acid esters, cholesterol, PEG-cholesterol, PEG-cholesterol derivatives, PEG-vitamin A, PEG-vitamin E, PEG-glycerol mono-(or di-)fatty acid esters, ethylene glycol mono-fatty acid esters, propylene glycol mono-fatty acid esters, 3-dialkyl(C1-8)amino-propylene glycol di-fatty acid esters, poly(ethylene glycol) mono-fatty acid esters, stearic acid, sorbitan esters, polyoxyethylene alkyl ethers, polyoxyethylene castor oil derivatives, polyoxyethylene sorbitan fatty acid esters, polyvinyl alcohol, polyvinylpyrrolidone, poloxamers, poloxamines, mixtures of sucrose stearate and sucrose distearate, random copolymers of vinyl acetate and vinyl pyrrolidone, deoxycholic acid, glycodeoxycholic acid, taurocholic acid, and the group consisting of anionic biopolymers (excluding casein or its derivative), anionic polymers, cationic biopolymers and mixtures thereof. In certain embodiments, the at least one surfactant is selected from the group of glycerol mono-(or di-)fatty acid esters excluding glycerol mono-stearate.

The present invention provides, in part, a solid powder containing fenofibrate nanoparticles, which has good stability, high concentration and which can be prepared at reasonable cost. Surprisingly, the fenofibrate nanoparticles can be stabilized by using the combination between casein or its derivative and glycerol mono-(or di-)fatty acid ester. The physical state of the fenofibrate nanoparticles is amorphous in majority as characterized by XRD. High concentration of fenofibrate in the prepared solid powder (20-35% by weight) may also facilitate the process transforming the powder into an orally administerable dosage form such as capsule, tablet, powder.

### Methods of Preparation

The particles in dry powder are characterized by SEM morphology analysis and XRD crystalline analysis. The dry powder is also re-dispersed in water and characterized for particle size distribution by Micromeritics Saturn DigiSizer 5200 using light scattering analysis technique.

The present invention provides, in part, nanoparticulate coenzyme Q10 compositions for pharmaceutical, nutraceutical and cosmetic use, and also for oral care use. The nanoparticulate coenzyme Q10 compositions are prepared according to the process described herein. About 1 part of coenzyme Q10 powder and about 0.05 to 5 parts of first surfactant are dissolved in about 1 to 100 parts of water miscible solvent or solvent mixture. Heating and homogenizing are applied to obtain clear solution in some cases. The mixture solution is heated to the temperature above the melting point (about 49-50° C.) of coenzyme Q10 but below the boiling point of the water miscible organic solvent. Then about 10 to 2000 parts of pre-heated water or aqueous solution with salt and/or additional surfactant is infused at flow rate between about 1 to 10,000 ml per minute into the coenzyme Q10 solution while homogenizing. After infusion, co-surfactant and optional bulking reagent are added, and the mixture is homogenized for additional time from about 0.5 to 10 minutes. The dispersed coenzyme Q10 suspension is dried by spray drying or freeze drying or rotovap evaporation or combination of them to yield nanoparticulate coenzyme Q10 dry powder.

The preferred water miscible organic solvent for preparation of nanoparticulate coenzyme Q10 compositions includes methanol, ethanol, 1-propanol, 2-propanol, formic acid, acetic acid, and mixtures thereof. The more preferred solvent includes ethanol, 1-propanol and acetic acid. The most preferred solvent is ethanol.

The preferred surfactant includes, but are not limited to, glycerol mono-(or di-)fatty acid esters, lecithin, phospholipids, cholesterol, PEG-phospholipids, PEG-cholesterol, PEG-cholesterol derivatives, PEG-vitamin A, PEG-vitamin E, PEG-glycerol mono-(or di-)fatty acid esters, ethylene glycol mono-fatty acid esters, propylene glycol mono-fatty acid esters, 3-dialkyl(C1-8)amino-propylene glycol di-fatty acid esters, poly(ethylene glycol) mono-fatty acid esters, stearic acid, sorbitan esters, polyoxyethylene alkyl ethers, polyoxyethylene castor oil derivatives, polyoxyethylene sorbitan fatty acid esters, polyvinyl alcohol, polyvinylpyrrolidone, poloxamers; poloxamines, mixtures of sucrose stearate and sucrose distearate, random copolymers of vinyl acetate and vinyl pyrrolidone, deoxycholic acid, glycodeoxycholic acid, taurocholic acid and mixtures thereof. The more preferred surfactant includes glycerol mono-(or di-)fatty acid esters, lecithin, phospholipids, PEG-phospholipids, PEG-vitamin E, PEG-glycerol mono-(or di-)fatty acid esters, ethylene glycol mono-fatty acid esters, propylene glycol mono-fatty acid esters, di-fatty acid esters, poly(ethylene glycol) mono-fatty acid esters. The most preferred surfactant is glycerol mono-(or di-)fatty acid ester, or lecithin, or phospholipids. The preferred co-surfactant added after infusion of water or aqueous solution includes but not limited to anionic biopolymers (such as casein or its derivative), anionic polymers, cationic biopolymers, and all preferred surfactants suitable for step (1). The most preferred co-surfactant is casein or its derivative.

The preferred bulking reagents include starches, and its derivatives, mannitol, lactose, maltitol, maltodextrin, maltose, dextrates, dextrin, dextrose, fructose, sorbitol, glucose, sucrose, carboxymethylcellulose, hydroxypropylcellulose, microcrystalline cellulose, ethylcellulose, methylcellulose, other suitable cellulose derivatives, gelatin, alginic acid, and its salt, colloidal silicon dioxide, croscarmellose sodium, crospovidone, magnesium aluminum silicate, povidone, benzyl phenylformate, chlorobutanol, diethyl phthalate, calcium stearate, glyceryl palmitostearate, magnesium oxide, poloxamer, polyvinyl alcohol, sodium benzoate, sodium lauryl sulfate, sodium stearyl fumarate, stearic acid, talc, zinc stearate, acacia, acrylic and methacrylic acid co-polymers, gums such as guar gum, pharmaceutical glaze, glyceryl palmitostearate, hydrogenated vegetable oil, kaolin, magnesium carbonate, magnesium oxide, polymethacrylates, sodium chloride, as well as other conventional bulking substances well known to persons skilled in the art. The most preferred bulking reagents are starch, sodium stearyl fumarate, stearic acid and other free flowing agents.

This nanoparticulate coenzyme Q10 compositions do not use large amount of diluents, can contain coenzyme Q10 at range of 1% to 50% by weight, preferably at 25% to 40%, and thus can be used with high concentrations. This nanoparticulate coenzyme Q10powder can be further processed into an orally administerable dosage form such as capsule, tablet, powder, and liquid beverage. The powder can also be processed into a cream for cosmetic use or a liquid dosage form for oral care.

The present invention also provides, in part, nanoparticulate fibrate compositions for pharmaceutical use. Fenofibrate is used as an example for the group of fibrate drugs which include bezafibrate, cipprofibrate, fenofibrate and gemfibrizol. The nanoparticulate fenofibrate compositions are prepared according to the process described herein. About 1 part of fenofibrate powder and about 0.05 to 5 parts of first surfactant are dissolved in about 1 to 100 parts of water miscible organic solvent. Heating and homogenizing are applied to obtain a clear solution in some cases. The mixture solution is heated to the temperature above the melting point (about 79-80° C.) of fenofibrate but below the boiling point of the water miscible organic solvent. Then about 5 to 2,000 parts of pre-heated water or aqueous solution with salt and/or additional surfactant is infused at flow rate between about 1 to 10,000 ml per minute into the fenofibrate solution while homogenizing. After infusion, co-surfactant and optional bulking reagent are added, and the mixture is homogenized for additional time from about 0.5 to 10 minutes. The dispersed fenofibrate nanoparticle suspension is dried by spray drying or freeze drying or rotovap evaporation or combination of them to yield nanoparticulate fenofibrate dry powder.

The preferred water miscible organic solvent for preparation of nanoparticulate fenofibrate compositions includes 1-propanol, formic acid, acetic acid, or mixture thereof. The preferred first surfactant includes glycerol mono-(or di-)fatty acid esters, lecithin, phospholipids, cholesterol, PEG-phospholipids, PEG-cholesterol, PEG-cholesterol derivatives, PEG-vitamin A, PEG-glycerol mono-(or di-)fatty acid esters, ethylene glycol mono-fatty acid esters, propylene glycol mono-fatty acid esters, 3-dialkyl(C1-8)amino-propylene glycol di-fatty acid esters, polyethylene glycol) mono-fatty acid esters, stearic acid, sorbitan esters, polyoxyethylene alkyl ethers, polyoxyethylene castor oil derivatives, polyoxyethylene sorbitan fatty acid esters, polyvinyl alcohol, polyvinylpyrrolidone, poloxamers; poloxamines, mixtures of sucrose stearate and sucrose distearate, random copolymers of vinyl acetate and vinyl pyrrolidone, deoxycholic acid, glycodeoxycholic acid, taurocholic acid and mixtures thereof. The more preferred surfactant includes glycerol mono-(or di-)fatty acid esters, lecithin, phospholipids, PEG-phospholipids, PEG-glycerol mono-(or di-)fatty acid esters, ethylene glycol mono-fatty acid esters, propylene glycol mono-(or di-)fatty acid esters, polyethylene glycol) mono-fatty acid esters. The most preferred surfactant is glycerol mono-(or di-)fatty acid esters.

The preferred co-surfactant added after infusion of water or aqueous solution includes anionic biopolymers (such as casein or its derivative), anionic polymers, cationic biopolymers, and all preferred surfactants described above. The most preferred co-surfactant is casein or its derivatives.

The preferred bulking reagents include starches, and its derivatives, mannitol, lactose, maltitol, maltodextrin, maltose, dextrates, dextrin, dextrose, fructose, sorbitol, glucose, sucrose, carboxymethylcellulose, hydroxypropylcellulose, microcrystalline cellulose, ethylcellulose, methylcellulose, other suitable cellulose derivatives, gelatin, alginic acid, and its salt, colloidal silicon dioxide, croscarmellose sodium, crospovidone, magnesium aluminum silicate, povidone, benzyl phenylformate, chlorobutanol, diethyl phthalate, calcium stearate, glyceryl palmitostearate, magnesium oxide, poloxamer, polyvinyl alcohol, sodium benzoate, sodium lauryl sulfate, sodium stearyl fumarate, stearic acid, talc, zinc stearate, acacia, acrylic and methacrylic acid co-polymers, gums such as guar gum, pharmaceutical glaze, glyceryl palmitostearate, hydrogenated vegetable oil, kaolin, magnesium carbonate, magnesium oxide, polymethacrylates, sodium chloride, as well as other conventional bulking substances well known to persons skilled in the art. The most preferred bulking reagents are starch, sodium stearyl fumarate, stearic acid and other free flowing agents.

This nanoparticulate fenofibrate compositions do not use large amount of diluents, can contain fenofibrate at range of 1% to 50% by weight, preferably at 20% to 40%, and thus can be used in high concentrations. This nanoparticulate fenofibrate compositions also contain high percentage of amorphous fenofibrate which is favorable to enhance oral bioavailability. The nanoparticulate fenofibrate powder can be further processed into an orally administerable dosage form such as capsule, tablet, powder for treating hyperlipidemia or hypercholesterolemia or both in a mammal, by providing an effective amount of each of fenofibrate and an excipient including casein or its derivatives.

The following examples are illustrative of the present invention. The present invention is not limited to the percentages, components and techniques described herein.

### EXAMPLES

### Example 1

### Preparation of Nanoparticulate Coenzyme Q10 Composition

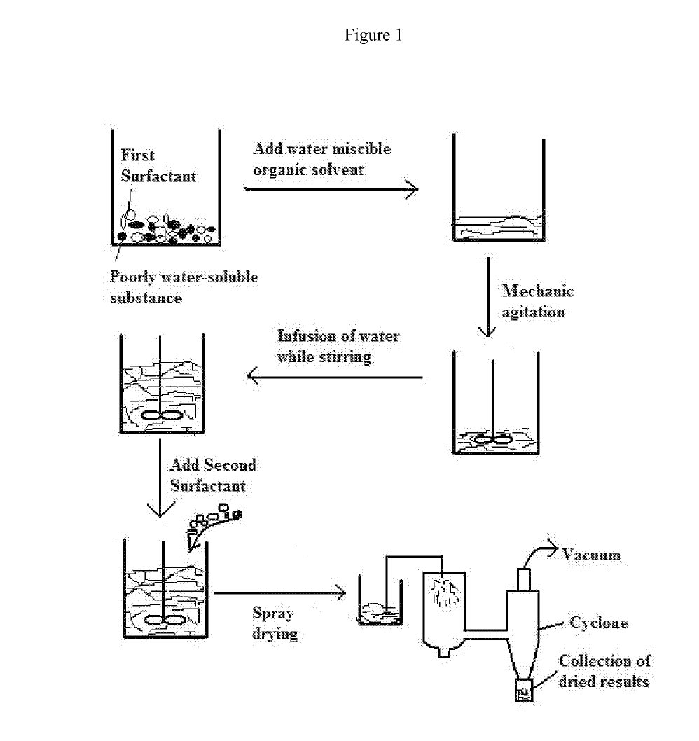

6.0 grams of coenzyme Q10 (Now Foods, Bloomingdale, Ill. 60108) and 3.0 gram of lecithin (California Academy of Health, Inc. CAOH, Temecula, Calif. 92592) are dissolved in 40 ml of ethanol by heating in a 60-65° C. water bath. While homogenizing with a mixer (Dynamic Mixer MD95, 2301 Sturgis Rd., Oxnard, Calif. 93030), 800 ml of 60-65° C. water is infused at flow rate of 50-100 ml per minute. After finished water infusion, 10.0 grams of sodium caseinate (cat# SLS2635, Sciencelab.com, Inc., 14025 Smith Rd., Houston, Tex. 77396) is added, and the resulting mixture is homogenized for additional 3 minutes in a 60-65° C. water bath, and then the mixture is spray-dried with Buchi 190 mini spray dryer with inlet temperature at 110° C. and outlet temperature at 65° C., and with Aspirator at full speed. The dry powder is subject to morphological analysis with scanning electronic microscope (shown in FIG. 1), characterized by XRD analysis, and also re-dispersed into water and analyzed by Micromeritics Saturn DigiSizer 5200 for particle size distribution. The crystallinity of the powder is about 21.1% as characterized by XRD analysis. The nanoparticle suspension is of median particle diameter at about 971 nm.

### Example 2

### Preparation of Nanoparticulate Fenofibrate Composition

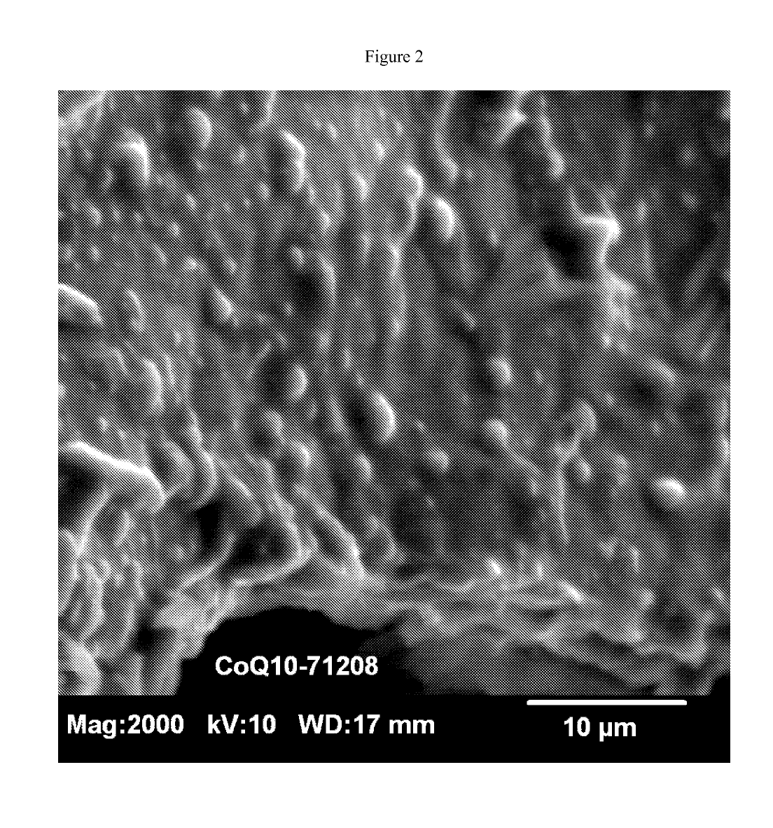

3 grams of fenofibrate (cat#SLF1921, Sciencelab.com, Inc.) and 1.5 gram of glycerol mono-oleate (Peceol, Gattefosse) are dissolved in 30 ml of 1-propanol by heating in a 80-85° C. water bath. While homogenizing with a mixer (Dynamic Mixer MD95), 600 ml of 80-85° C. water is infused at flow rate of 15-60 ml per minute. After finished water infusion, 6.0 grams of sodium caseinate (cat# SLS2635, Sciencelab.com, Inc.) is added, and the resulting mixture is homogenized for additional 3-5 minutes in an 80-85° C. water bath, and then the mixture is spray-dried with Buchi 190 mini spray dryer with inlet temperature at 110° C. and outlet temperature at 75° C., and with Aspirator at full speed. The dry powder is subject to morphological analysis with scanning electronic microscope (shown in FIG. 2), characterized by XRD analysis, and also re-dispersed into water and analyzed by Micromeritics Saturn DigiSizer 5200 for particle size distribution. The crystallinity of the powder is about 29.9% as characterized by XRD analysis. The nanoparticle suspension is of median particle diameter at about 820 nm.

What is claimed is:

1. A method for the preparation of nanoparticles containing a poorly water-soluble pharmaceutically active compound, which method comprises:
mixing the compound and at least one surfactant in a water-miscible organic solvent to form a solution;
infusing water and optionally an additional surfactant to the solution while homogenizing the solution to form a suspension;
optionally adding at least one co-surfactant and/or bulking agent to the suspension while homogenizing the suspension; and
drying the suspension to provide nanoparticles containing the poorly water-soluble pharmaceutically active compound having a particle size in the range from about 50 nm to about 5000 nm, wherein said drying is achieved by spray drying, roto-vap evaporation, or freeze drying.

2. The method of claim 1 wherein the water-miscible organic solvent is selected from the group consisting of acetic acid, acetone, methanol, ethanol, 1-propanol, 2-propanol, formic acid, propionic acid, and mixtures thereof

3. The method of claim 1 wherein the at least one surfactant is selected from the group consisting of glycerol mono-(or di-)fatty acid esters, lecithin, phospholipids (such as phosphatidyl choline, phosphatidyl ethanolamine, phosphatidyl inositol, sphingomyelin, and the like), cholesterol, PEG-phospholipids, PEG-cholesterol, PEG-cholesterol derivatives, PEG-vitamin A, PEG-vitamin E, PEG-glycerol mono-(or di-)fatty acid esters, ethylene glycol mono-fatty acid esters, propylene glycol mono-fatty acid esters, 3-dialkyl(C1-8)amino-propylene glycol di-fatty acid esters, poly(ethylene glycol) mono-fatty acid esters, stearic acid, sorbitan esters, polyoxyethylene alkyl ethers, polyoxyethylene castor oil derivatives, polyoxyethylene sorbitan fatty acid esters, polyvinyl alcohol, polyvinylpyrrolidone, poloxamers; poloxamines, mixtures of sucrose stearate and sucrose distearate, random copolymers of vinyl acetate and vinyl pyrrolidone, deoxycholic acid, glycodeoxycholic acid, taurocholic acid and mixtures thereof.

4. The method of claim 1 wherein the additional surfactant and the co-surfactant are each independently selected from the group consisting of: anionic biopolymers (such as casein or its derivative), anionic polymers, cationic biopolymers and mixtures thereof.

5. The method of claim 1 wherein the bulking agent is selected from starchesor its derivatives, mannitol, lactose, maltitol, maltodextrin, maltose, dextrates, dextrin, dextrose, fructose, sorbitol, glucose, sucrose, carboxymethylcellulose, hydroxypropylcellulose, microcrystalline cellulose, ethylcellulose, methylcellulose, other suitable cellulose derivatives, gelatin, alginic acid, and its salt, colloidal silicon dioxide, croscarmellose sodium, crospovidone, magnesium aluminum silicate, povidone, benzyl phenylformate, chlorobutanol, diethyl phthalate, calcium stearate, glyceryl palmitostearate, magnesium oxide, poloxamer, polyvinyl alcohol, sodium benzoate, sodium lauryl sulfate, sodium stearyl fumarate, stearic acid, talc, zinc stearate, acacia, acrylic and methacrylic acid co-polymers, gums such as guar gum, milk derivatives such as whey, pharmaceutical glaze, glyceryl palmitostearate, hydrogenated vegetable oil, kaolin, magnesium carbonate, magnesium oxide, polymethacrylates, sodium chloride and mixtures thereof.

6. The method of claim 1 wherein the poorly water-soluble pharmaceutically active compound is coenzyme Q10; wherein the at least one surfactant is a phospholipid; wherein the additional surfactant and the co-surfactant are each independently sodium caseinate.

7. The method of claim 1 wherein the poorly water-soluble pharmaceutically active compound is fenofibrate ; wherein the at least one surfactant is glycerol mono-oleate; wherein the additional surfactant and the co-surfactant are each independently sodium caseinate.

8. A composition comprising:
about 1-60% by weight nanoparticles of a pharmaceutically active compound;
about 5-90% by weight at least one surfactant and casein or its derivative co-surfactant which are on the surface of the nanoparticles;
about 0-90% by weight a bulking agent; and
about 0-5% by weight water.

9. The composition of claim 8 wherein the at least one surfactant is selected from glycerol mono-(or di-)fatty acid esters, lecithin, phospholipids, and mixtures thereof

10. The composition of claim 8, comprising:
about 1-60% by weight nanoparticles of coenzyme Q10;
about 5-90% by weight at least one surfactant and at least one co-surfactant which are on the surface of the nanoparticles;
about 0-90% by weight a bulking agent; and
about 0-5% by weight water.

11. The composition of claim 10 wherein the coenzyme Q10 is in a form selected from the group consisting of a crystalline phase, an amorphous, a semi-crystalline phase, a semi-amorphous, and mixtures thereof

12. The composition of claim 10 wherein the size of the nanoparticles is in the range selected from the group consisting of less than about 5 μm, less than about 3 μm, less than about 1.5 μm, less than about 1 μm, less than about 900 nm, less than about 800 nm, less than about 700 nm, less than about 600 nm, less than about 500 nm, less than about 400 nm, less than about 300 nm, less than about 200 nm, and less than about 100 nm.

13. The composition of claim 10 wherein the at least one surfactant is selected from the group consisting of glycerol mono-(or di-)fatty acid esters, lecithin, phospholipids and mixtures thereof; and the at least one co-surfactant is casein or its derivatives.

14. The composition of claim 10 wherein the at least one surfactant is lecithin and the at least one co-surfactant is sodium caseinate.

15. A composition comprising;
about 5-60% by weight nanoparticles of fibrate;
about 5-90% by weight at least one surfactant and at least one co-surfactant which are on the surface of the nanoparticles; and
about 0-90% by weight a bulking agent; and
about 0-5% by weight water.

16. The composition of claim 15 wherein the fibrate is fenofibrate.

17. The composition of claim 16 wherein over 50% of the fenofibrate is in a form of amorphous phase.

18. The composition of claim 15 wherein the size of the nanoparticles is in the range selected from the group consisting of less than about 5 μm, less than about 3 μm, less than about 1.5 μm, less than about 1 μm, less than about 900 nm, less than about 800 nm, less than about 700 nm, less than about 600 nm, less than about 500 nm, less than about 400 nm, less than about 300 nm, less than about 200 nm, and less than about 100 nm.

19. The composition of claim 15 wherein the at least one co-surfactant is casein or its derivatives; and wherein the at least one surfactant is selected from glycerol mono-(or di-) fatty acid esters, cholesterol, PEG-cholesterol, PEG-cholesterol derivatives, PEG-vitamin A, PEG-vitamin E, PEG-glycerol mono-(or di-)fatty acid esters, ethylene glycol mono-fatty acid esters, propylene glycol mono-fatty acid esters, 3-dialkyl(C1-8)amino-propylene glycol di-fatty acid esters, poly(ethylene glycol) mono-fatty acid esters, stearic acid, sorbitan esters, polyoxyethylene alkyl ethers, polyoxyethylene castor oil derivatives, polyoxyethylene sorbitan fatty acid esters, polyvinyl alcohol, polyvinylpyrrolidone, poloxamers, poloxamines, mixtures of sucrose stearate and sucrose distearate, random copolymers of vinyl acetate and vinyl pyrrolidone, deoxycholic acid, glycodeoxycholic acid, taurocholic acid, and the group consisting of anionic biopolymers (excluding casein or its derivative), anionic polymers, cationic biopolymers and mixtures thereof

20. The composition of claim 15 wherein the at least one surfactant is selected from the group of glycerol mono-(or di-)fatty acid esters excluding glycerol mono-stearate.
